(19) United States
(12) Patent Application Publication
(10) Pub. No.: US 2003/0119728 A1
(43) Pub. Date: Jun. 26, 2003

(54) Stable, nasally, orally or sublingually applicable pharmaceutical preparation

(76) Inventors: Helmut Scheidl, Brunnau Fieberbrunn (AT); Gerhard Hantich, Stockerdorfl Kitzbuhel (AT); Ernst Hesse, Reitliftweg Fieberbrunn (AT); Thomas Zapf, Bonn (AT)

Correspondence Address: JACOBSON HOLMAN PLLC, 400 SEVENTH STREET N.W., SUITE 600, WASHINGTON, DC, 20004, US

(21) Appl. No.: 10/203,614

(22) PCT Filed: Jan. 10, 2001
(86) PCT No.: PCT/AT01/00007

(30) Foreign Application Priority Data
Feb. 16, 2000 (AT) A 233/2000

(51) Int. Cl. A61K 38/12

(52) U.S. Cl. 514/11

(57) ABSTRACT

A stable, nasally, orally or sublingually applicable pharmaceutical preparation for administering to patients has an aqueous solution of desmopressin as the active agent. Said solution contains an osmoticum and a buffer which maintains the pH-value in the range of 4 to 6, preferably about 5. The buffer is malic acid, preferably in the form of a racemate. Thereby an improved stability of the desmopressin content in the preparation is obtained.

A stable pharmaceutical preparation for nasal, oral or sublingual administration

The invention relates to a stable pharmaceutical preparation for nasal, oral or sublingual administration to patients in the form of a liquid solution, in particular an aqueous solution of desmopressin as the active substance, this liquid solution containing an osmotic agent and a buffer which maintains the pH within the range 4 to 6, preferably at around 5.

Desmopressin (1-deamino-8-D-arginine-vasopressin) is a peptide hormone with high therapeutic efficacy. In liquid pharmaceutical preparations it is therefore as a rule present in a low concentration. To ensure the efficacy of the preparation, stabilisation must be performed in order to minimise chemical and microbial degradation. U.S. Pat. No. 5,482,931 or WO 95/01185 have already proposed the use of benzalkonium chloride as the preservative, and the use of a suitable buffer which maintains the pH of the aqueous composition between 4 to 6, preferably at around 5. With this arrangement, the best stabilisation of desmopressin can be achieved with the use of acetate as the buffer. This is unsatisfactory in practice, however, as acetic acid has an unpleasant odour. The above-mentioned publications therefore also propose a citrate-phosphate buffer system, in all cases in combination with benzalkonium chloride as the preservative, which also allegedly prevents adsorption on to the vessel walls.

The object of the invention is to further improve the stabilisation of the active substance desmopressin in a pharmaceutical preparation of the type described in the introduction, achieving this independently of the use or the type of preservative.

Surprisingly, investigations have shown that the target advantages can be achieved without problems by the use of malic acid as the buffer, without disadvantages of another kind having to be taken into account. A pharmaceutical preparation of the kind according to the invention therefore contains desmopressin as the active substance—in particular in a low concentration —, malic acid, which is used to stabilise the desmopressin and as a buffer to adjust the pH to within the range 4 to 6, preferably to around 5, and a suitable additive as the osmotic agent. In the context of the present invention, the malic acid thus has a dual function: on the one hand it forms the buffer for adjustment of the pH, and on the other it ensures stabilisation of the desmopressin.

The pharmaceutical use of the preparation according to the invention is mainly for the treatment of antidiuretic disturbances, in particular enuresis nocturna and diabetes insipidus. The treatment of haemorrhagic diseases, such as e.g. haemophilia A, Willebrand-Jürgen's syndrome and postoperative bleeding, is also possible.

As a rule it is sufficient to use the malic-acid buffer in a low concentration, preferably within the range 1 to 5 mM, in particular at around 2.5 mM. When used in this concentration, the malic acid may be present as the racemate, which is financially advantageous, but the D- or L-form may also be used either alone, in combination with each other, or in combination with the racemate.

Sodium chloride is known to be a suitable osmotic agent.

Other buffers may also be used in addition to malic acid, e.g. acetate/acetic acid, without impairing the advantages of malic acid.

It is especially advantageous that the preparation according to the invention can be kept free from preservatives without stabilisation of the active substance desmopressin being thereby impaired. The introduction of micro-organisms into the desmopressin solution in the malic-acid system can be prevented by aseptic decanting and/or by the addition of antimicrobial substances.

The preparation according to the invention is suitable for nasal, oral or sublingual administration.

Further characteristics and advantages of the invention are revealed in the description of the following embodiments or comparison studies:

Here the following solutions of desmopressin acetate were used as formulations:

| | Desmopressin acetate in | | Buffer |
|---|---|---|---|
| Formulation No. | mg/ml | Preservative in mg/ml | [conc. in mM] |
| 1 | 0.100 | Benz. 0.10 | DL-malic acid [2.5] |
| 2 | 0.10 | Benz. 0.10 | Malic acid [25] |
| 3 | 0.10 | Benz. 0.10 | Citric acid and Na₂HPO₄ |
| | | | [tog. 25] |
| 4 | 0.10 | Benz. 0.10 | NaH₂PO₄ [19] |
| | | | pH = 1.0 |
| 5 | 0.11 | Benz. 0.10 | NaH₂PO₄ [19] |
| | | | pH = 2.0 |
| 6 | 0.10 | Benz. 0.10 | NaH₂PO₄ [19] |
| | | | pH = 3.0 |
| 7 | 0.11 | Benz. 0.10 | NaH₂PO₄ [19] |
| | | | pH = 4.0 |
| 8 | 0.10 | Benz. 010 | KH₂PO₄ and Na₂HPO₄ |
| | | | [tog. 19] |
| | | | pH = 5.0 |
| 9 | 0.10 | Benz. 0.13 | Citric acid [60] |
| | | | pH = 6.0 |
| 10 | 0.10 | Benz. 0.13 | KH₂PO₄ and Na₂HPO₄ |
| | | | [tog. 67] |
| | | | pH = 7.0 |
| 11 | 0.10 | Benz. 0.10 | L-malic acid [2.5] |
| 12 | 0.10 | Benz. 0.10 | D-malic acid [2.5] |
| 13 | 0.10 | Benz. 0.10 | Malic acid [2.5] |
| 14 | 0.10 | Benz. 0.10 | Malic acid/NaAc: [2.5] |
| 15 | 0.10 | — | Malic acid [2.5] |
| 16 | 0.02 | Benz. 0.10 | Malic acid [2.5] |
| 17 | 2.00 | Benz. 0.10 | Malic acid [2.5] |
| 18 | 0.10 | Benz. 0.10 | Malic acid [1.0] |
| 19 | 0.10 | Benz. 0.10 | Malic acid [5.0] |
| 20 | 0.10 | Benz. 0.05 | Malic acid [2.5] |
| 21 | 0.10 | Benz. 0.20 | Malic acid [2.5] |
| 22 | 0.10 | p-Hydroxyb. 2.0 | Malic acid [2.5] |
| 23 | 0.10 | — | 80% malic acid [2.5] and |
| | | | 20% Cit/PO₄³⁻ [25] |
| 24 | 0.10 | — | 60% malic acid [2.5] and |
| | | | 40% Cit/PO₄³⁻ [25] |
| 25 | 0.10 | — | 50% malic acid [2.5] and |
| | | | 50% Cit/PO₄³⁻ [25] |
| 26 | 0.10 | — | 40% malic acid [2.5] and |
| | | | 60% Cit/PO₄³⁻ [25] |
| 27 | 0.10 | — | 20% malic acid [2.5] and |
| | | | 80% Cit/PO₄³⁻ [25] |

The abbreviations used in this table and the table below are as follows:
Cit/PO₄³⁻—Citrate phosphate
Ac—Acetate
HAc—Acetic acid
Benz—Benzalkonium chloride
NH₄Ac—Ammonium acetate
p-Hydroxyb.—p-hydroxybenzoic acid methyl ester
mM—millimol/litre

The desmopressin solutions (1 litre in each case) used in the experiments below were generally prepared by the following method:

a) 989.15 g distilled water for injection was weighed out into a 1-litre glass beaker;

b) Of this, about 30 g distilled water for injection was poured into a glass beaker for rinsing;

c) 9.115 g very pure sodium chloride Ph.Eur. and the buffer used (in the case of malic acid 0.335 g) were dissolved in the remaining distilled water from a) while stirring with a magnetic stirrer. The weighing vessels were rinsed in each case with approximately 5 g distilled water from b);

d) With stirring, the quantity of desmopressin acetate 100% used in each case (weighed portion with respect to the content by weight) was added, and the weighing vessel was rinsed twice with approximately 5 g distilled water;

e) If necessary, the quantity of preservatives used in each case (usually benzalkonium chloride 100% (weighed portion with respect to the actual weight) was added, and the weighing vessel was rinsed twice with approximately 5 g distilled water; this was followed by stirring for approximately ½ h.

f) The pH was adjusted to the value valid for the particular formulation (at most 5.0±0.2) with approximately 4.2 ml 1 N NaOH solution;

g) 1003.0 g of the final solution corresponds to 1000 ml;

h) Sterile filtration of the final solution was performed with a millipak sterile filter.

The substances used in each particular case were obtained from the following manufacturers:

| Substance | Manufacturer |
|---|---|
| Desmopressin acetate | UCB Belgien |
| Benzalkonium chloride | Ferrosan |
| p-hydroxybenzoic acid methyl ester | Merck Darmstadt |
| NaCl | Österr. Salinen AG or Merck |
| HCl | 1 N (cat. No. 10448) Merck |
| | Darmstadt |
| NaOH | Platzchen, Merck Darmstadt |
| DL-malic acid | Merck Darmstadt |
| Acetic acid | 100% ultra pure, Merck Darmstadt |
| Millipak filter 0.22 μm | Millipore |
| (Durapore ®: PVDF) | |

Comparative Study of the Stability of Desmopressin:

The study was conducted with the use on the one hand of a solution according to the prior art (formulation No. 3), and on the other a formulation according to the invention (formulation No. 1). The two formulations were in each case stored at 25° C. and 50° C. for a period of 10 months in each case, and then analysed for the content of the degradation products G1, G2, G3 and G4. The degradation products are as follows

| | |
|---|---|
| G1 | 5-asparaginic acid desmopressin |
| G2 | 4-glutaminic acid desmopressin |
| G3 | 9-glycine desmopressin |
| G4 | isomer of 5-asparaginic acid desmopressin |

The values indicated in the table below give the degradation products, with reference to desmopressin (in % A/A, i.e. area percent with reference to the active substance):

| Formulation | 10 months/50° C. | | | | 10 months/25° C. | | | |
|---|---|---|---|---|---|---|---|---|
| No. | G4 | G3 | G1 | G2 | G4 | G3 | G1 | G2 |
| 3 | 5.27 | 1.85 | 1.16 | 2.44 | 0.26 | 0.18 | 0.07 | 0.21 |
| 1 | 3.45 | 1.19 | 0.95 | 1.48 | 0.14 | 0.11 | 0.05 | 0.13 |

As is apparent from the comparison of the values obtained for the four degradation products of desmopressin, the malic-acid buffer system results in much higher stability of the active substance desmopressin than the citrate-phosphate buffer system, both after storing the solutions at room temperature and under conditions of stress.

Desmopressin Stability in the Malic-acid Buffer System:

Preliminary studies revealed that desmopressin is at its most stable in the region of pH=5.0. Desmopressin preparations with various pH values (1.0, 2.0, 3.0, 4.0, 5.0, 6.0, and 7.0) were investigated for these preliminary studies. These solutions were stored in glass flasks for 6 weeks at 50° C. and then analysed in the usual way with the use of analytical columns. After 6 weeks the pH values of the seven solutions were unchanged. The investigations for the contents of desmopressin and sum of the areas of the previously mentioned degradation products G1, G2, G3 and G4 revealed that desmopressin is most stable in the region of pH=5.0.

Isotonic desmopressin solutions according to formulations 1 and 2 were therefore investigated, these formulations therefore exhibiting malic acid as the buffer in the concentration 2.5 mM and 25 mM respectively. The pH of both preparations was 5.0. The two desmopressin solutions were stored in glass flasks for two months at 40° C. This was followed by the determination of desmopressin and the degradation products 9-glycine desmopressin (G3) and 5-argininic acid desmopressin (G1).

The result is expressed in the table below as the ratio of the degradation product to desmopression, standardised to the formulation with the smallest mass ratio.

| Formulation No. | Malic-acid concentrate | G1 | G3 |
|---|---|---|---|
| 1 | 2.5 | 1.0 | 1.0 |
| 2 | 25.0 | 1.9 | 1.3 |

As can be seen from the data, desmopressin in the more dilute malic-acid buffer surprisingly shows a considerably smaller quantity of degradation products, i.e. a higher stability.

Desmopressin Stability as a Function of the Chiral Form of Malic Acid:

The formulations Nos. 1, 11 and 12 were prepared. These solutions were analysed after 2 and 4 weeks of storage at 65° C. in glass vessels of hydrolytic class I for the desmopressin content and the quantity of degradation products. The following results were obtained:

| | | Desmopressin concentration in | | Sum of the degradation products | |
|---|---|---|---|---|---|
| Formulation | Type of malic | μg/ml | | in % A/A after | |
| No. | acid | 2 weeks | 4 weeks | 2 weeks | 4 weeks |
| 1 | DL | 94.9 | 93.2 | <0.2 | 0.72 |
| 11 | L | 94.6 | 93.4 | 0.2 | 0.65 |
| 12 | D | 95.1 | 93.2 | <0.2 | 0.54 |

The sum of the degradation products comprises:

5-asparaginic acid desmopressin,

4-glutaminic acid desmopressin,

9-glycine desmopressin,

Isomeric 5-asparaginic acid desmopressin.

As shown by the data, the chiral form of malic acid plays no role in the stability of desmopressin.

Influence of the Preservative Benzalkonium Chloride on the Stability of Desmopressin:

Formulations 13, 14 and 15 were compared with one another under study conditions, the DL-malic acid in formulation 14 being reduced by 20% mol/mol and replaced with the corresponding quantity of acetate buffer. The procedure was as follows:

The solutions were stored at 65° C. for 7 weeks, and analysed during this period (after 1, 2, 3, 5, 7 weeks) for the desmopressin content and the content of the degradation products (G1, G2, G3, G4).

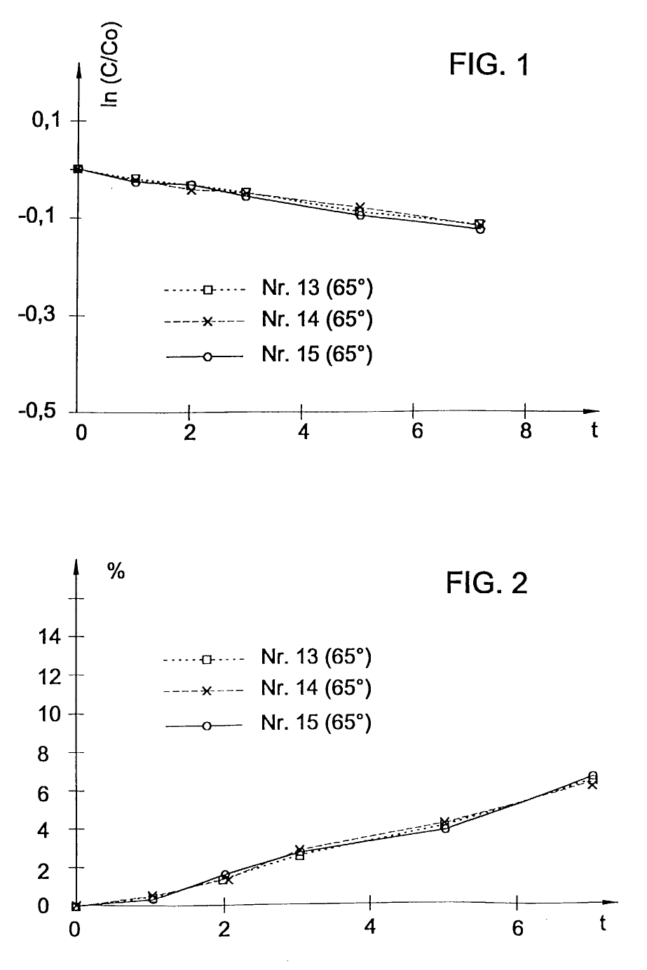

FIG. 1 shows the decrease in the desmopressin content, and FIG. 2 the increase in the sum of the degradation products G1 to G4, in both figures time t being plotted in weeks on the x-axis. The value In c/cₒ is plotted on the y-axis in FIG. 1, and the degradation products in % with reference to desmopressin in FIG. 2.

The table below shows the calculated rate constants, wherein:

1n c/cₒ=−kt;

t=7 weeks=4233600 s;

T=65° C.

| Formulation No. | Composition | K 65 C/^{s-1} · 10⁻⁸ |
|---|---|---|
| 13 | Malic acid with benz. | 2.98 |
| 14 | Malic acid/acetic acid + benz. | 2.93 |
| 15 | Malic acid without benz. | 3.07 |

From the above results it can be concluded that the improved stabilisation of desmopressin is due to the malic-acid buffer and not to the presence of benzalkonium chloride, for the difference between the results in formulations 13, 14, and 15 is so small that it lies within the experimental margin of error and is therefore without importance.

From the above results it can furthermore be concluded that even in the presence of another buffer substance (such as e.g. acetate), the malic acid stabilises the desmopressin better than previously conventional buffer systems were able to do.

Study of the Stability of Desmopressin in the Malic-acid Buffer System by Comparison with the Citrate Phosphate Buffer System:

In this study a solution according to formulation No. 1 was compared with a formulation No. 3, a series of mixtures of these two formulations having been produced with differing mixing ratios. These mixtures were stored for 4 weeks at 65° C. and, after storing for 2 and 4 weeks, analysed for the content of desmopressin and its degradation products (G1 to G4).

The results are summarised in the two tables below, the values for the secondary peaks in % (A/A) with reference to desmopressin, the values for desmopressin in μg/ml (corresp. to % of the reference value).

The meanings for the degradation products G1 to G4 are the same as those mentioned above.

Values after 2 weeks at 65° C.:

| Mixing ratio | | | | | | | |
|---|---|---|---|---|---|---|---|
| DL-malic acid | | | | | | | |
| (form No. 1) to | | | | | Unknown | Sum of | |
| citrate/phosphate | | | | | secondary | all | |
| (form. No. 3) | G3 | G1 | G2 | G4 | peaks | sec. peaks | Desmopressin |
| 100:0 | 0.31 | 0.29 | 0.41 | 0.85 | 0 | 1.86 | 94.85 |
| 80:20 | 0.4 | 0.41 | 0.51 | 1.28 | 0.41 | 3.01 | 94.39 |
| 60:40 | 0.46 | 0.44 | 0.57 | 1.43 | 0.51 | 3.41 | 93.3 |
| 50:50 | 0.46 | 0.43 | 0.58 | 1.42 | 0.54 | 3.43 | 93.28 |
| 40:60 | 0.41 | 0.4 | 0.53 | 1.29 | 0.32 | 2.95 | 93.24 |
| 20:80 | 0.49 | 0.45 | 0.64 | 1.47 | 0.65 | 3.7 | 92.9 |
| 0:100 | 0.5 | 0.43 | 0.67 | 1.43 | 0.65 | 3.68 | 92.28 |

Values after 4 weeks at 65° C.:

| Mixing ratio | | | | | | | |
|---|---|---|---|---|---|---|---|
| DL-malic acid | | | | | | | |
| (form No. 1) to | | | | | Unknown | Sum of | |
| citrate/phosphate | | | | | secondary | all | |
| (form. No. 3) | G3 | G1 | G2 | G4 | peaks | sec. peaks | Desmopressin |
| 100:0 | 0.64 | 0.5 | 0.64 | 1.62 | 0.72 | 4.12 | 93.21 |
| 80:20 | 0.66 | 0.62 | 0.73 | 2.11 | 1.23 | 5.35 | 92.20 |
| 60:40 | 0.75 | 0.7 | 0.85 | 2.37 | 1.31 | 5.98 | 91.26 |
| 50:50 | 0.69 | 0.66 | 0.83 | 2.18 | 1.34 | 5.7 | 91.43 |
| 40:60 | 0.64 | 0.61 | 0.78 | 2.07 | 1.49 | 5.59 | 91.53 |
| 20:80 | 0.79 | 0.72 | 0.95 | 2.37 | 1.49 | 6.32 | 89.97 |
| 0:100 | 0.84 | 0.74 | 0.99 | 2.34 | 1.69 | 6.6 | 89.74 |

From the above tables it is apparent that the stabilisation of desmopressin increases by mixing malic-acid buffer into the citrate/phosphate buffer. This is observed at the higher desmopressin content and at the lower content of desmopressin degradation products after 2 and 4 weeks of storage at 65° C. Thus this study too demonstrates the better suitability of the malic-acid buffer as compared with known systems for the chemical stabilisation of desmopressin in solution.

Study of the Stability of Desmopressin in Differing Concentrations:

In order to test various concentrations of the active substance (desmopressin), formulation No. 1 was modified in that the content of 0.100 mg/ml desmopressin acetate was replaced with a content of 2.00 mg/ml (formulation No. 17), or by a content of 0.02 mg/ml (formulation 16). The solutions thus prepared were stored for 4 weeks at 65° C. and in each case analysed after 2 and 4 weeks for the content of desmopressin and its degradation products (G1 to G4), expressed as a total area.

| | | | Sum of the degradation products G1-G4 | |
|---|---|---|---|---|
| | Desmopressin acetate (% of the | | (in % A/A, with reference to desmopressin | |
| | reference value) after | | acetate) after | |
| Formulation No. | 2 weeks | 4 weeks | 2 weeks | 4 weeks |
| 17 | 96.6 | 91.6 | 1.76 | 4.03 |
| 16 | 95.9 | 92.7 | 7.9 | 8.7 |

As the results show, the formulations are sufficiently stable to enable one to vary the desmopressin acetate content within a certain framework without the stability of desmopressin being substantially impaired as a result.

Investigation of the Stability of Desmopressin in Variously Concentrated Malic-acid Solutions:

In order to test the influence of the concentration of the buffer (malic acid), formulation No. 1 was modified in such a way that the concentration of the malic-acid content (2.5 mM) in formulation No. 1 was replaced with a concentration of 1.0 mM (formulation No. 18) or 5.0 mM (formulation No. 19).

The solutions thus prepared were stored for 4 weeks at 65° C. and in each case analysed after 2 and 4 weeks for the content of desmopressin and its degradation products (G1 to G4), expressed as a total area.

The following values were obtained:

| | | | Sum of the degradation products G1-G4 | |
|---|---|---|---|---|
| | Desmopressin acetate (% of the | | (in % A/A, with reference to desmopressin | |
| | reference value) after | | acetate) after | |
| Formulation No. | 2 weeks | 4 weeks | 2 weeks | 4 weeks |
| 18 | 97.3 | 95.3 | 1.86 | 3.9 |
| 19 | 96.9 | 95.4 | 1.93 | 4.27 |

As the results show, the formulation is sufficiently stable for the concentration of the malic-acid buffer to be varied within a certain framework without substantially losing the stability of the desmopressin content.

Investigation of the Stability of Desmopressin in the Presence of Various Concentrations of Benzalkonium Chloride:

In order to test the influence of the concentration of the preservative benzalkonium chloride, formulation No. 1 was modified in such a way that the content of 0.100 mg/ml benzalkonium chloride was replaced with a concentration of 0.20 mM (formulation No. 21) or 0.05 mM (formulation No. 20).

The solutions thus prepared were stored for 4 weeks at 65° C. and in each case analysed after 2 and 4 weeks for the content of desmopressin and its degradation products (G1 to G4), expressed as a total area.

The following values were obtained:

| | | | Sum of the degradation products G1-G4 | |
|---|---|---|---|---|
| | Desmopressin acetate (% of the | | (in % A/A, with reference to desmopressin | |
| | reference value) after | | acetate) after | |
| Formulation No. | 2 weeks | 4 weeks | 2 weeks | 4 weeks |
| 21 | 96.9 | 94.8 | 1.90 | 3.97 |
| 20 | 98.5 | 94.7 | 1.89 | 4.09 |

As the results show, the formulation is sufficiently stable to enable one to vary the content of the preservative benzalkonium chloride, if a content of this substance is in fact required, within a certain framework without the stability of the formulation being substantially impaired.

Investigation of the Stability of Desmopressin in the Presence of p-hydroxybenzoic Acid Methyl Ester as the Preservative:

For this purpose formulation No. 22 was prepared which, in place of benzalkonium chloride, contains p-hydroxybenzoic acid methyl ester with a content of 0.2% as the preservative.

The solution thus prepared was stored for 4 weeks at 65° C. and analysed after 2 and 4 weeks for the content of desmopressin and its degradation products (G2 and G3), expressed as a total area.

The following values were obtained:

| | | | Sum of the degradation products G1-G4 | |
|---|---|---|---|---|
| | Desmopressin acetate (% of the | | (in % A/A, with reference to desmopressin | |
| | reference value) after | | acetate) after | |
| Formulation No. | 2 weeks | 4 weeks | 2 weeks | 4 weeks |
| 22 | 92.7 | 88.0 | 1.51 | 3.0 |

When compared with the results for benzalkonium chloride as the preservative, the results show that when the latter cannot be used, perhaps for reasons of tolerability, this preservative can be replaced with another one.

Calculation of the Possible Shelf Life:

As mentioned earlier, the rate constant for the degradation of desmopressin in the buffer system according to the invention (formulations Nos. 1, 13) 2.98·10⁸ s⁻¹ in the presence of benzalkonium chloride and in the absence of the preservative (formulation No. 15) 3.07·10⁸ s⁻¹.

The rate constant of the formulation A described in U.S. Pat. No. 5,482,931 is 4.6·10⁸ s⁻¹. Thus the rate of degradation of desmopressin in the malic-acid system is lower by a factor of 1.5, independently of the presence or absence of benzalkonium chloride. This means that the stability and thus the possible shelf life of the preparation according to the invention is about 50% higher than a preparation in accordance with the above-mentioned formulation A.

By comparison with the formulation B mentioned in the above published patent: the rate constant of the degradation of desmopressin for formulation B is 8.0·10⁸ s⁻¹. Thus the stability of desmopressin in the malic-acid system is 2.6 times higher than in the citrate-phosphate system—independently of the presence or absence of benzalkonium chloride. This results in actually more than twice the shelf life in the buffer system according to the invention.

In accordance with the invention desmopressin can be used in a commercially available form, i.e. in the pure form or in the form of its salts, e.g. as the acetate. Similarly the malic acid can be used in the commercially available form, i.e. in the pure form or in the form of its common salts, e.g. as the sodium salt. As the pharmaceutical preparation according to the invention is liquid, the malic acid is always available in a dissolved form.

Although water is by far the most commonly used solvent, other solvents, in particular alcohol or mixtures of water with other solvents, can also be used. Similarly, the preparation according to the invention may contain residues in small quantities.

The following examples illustrate the nature of the invention:

### EXAMPLE 1

A nasal spray without preservative for the treatment of antidiuretic disturbances and haemorrhagic diseases is prepared by pouring 4900 g water for injection into a 5-l glass beaker and dissolving 45.58 g sodium chloride, 1.675 g malic acid and 0.5 g desmopressin acetate therein with stirring. The pH is adjusted to pH 5 with 1 N NaOH. The solution is made up to 5 l with water for injection and, under aseptic conditions, decanted through a sterile millipak filter into small brown-glass bottles of hydrolytic class I, and sealed with sterile pump heads and suitable nasal adaptors.

The preparation and decanting are performed in pharmaceutical clean rooms under aseptic conditions.

### EXAMPLE 2

A nasal spray with benzalkonium chloride as the preservative for the treatment of antidiuretic disturbances and haemorrhagic diseases is prepared by pouring 990 g water for injection into a 1-litre glass beaker and dissolving 9.115 g sodium chloride, 0.1 g desmopressin acetate, 0.1 g benzalkonium chloride and 0.335 g malic acid therein. The pH is adjusted to pH 5 with approximately 4.2 ml 1 N NaOH. The solution is made up to 1 litre, filtered through a Millipak filter, decanted into small brown-glass bottles, and sealed with the pump caps.

The preparation and decanting are performed in pharmaceutical production rooms under germ-free conditions.

### EXAMPLE 3

A low-concentration sublingual spray for the treatment of antidiuretic disturbances and haemorrhagic diseases is prepared as follows: 9900 g water for injection is poured into a suitable glass beaker and 4 g desmopressin acetate, 1 g benzalkonium chloride, 91.15 g sodium chloride and 3.35 g malic acid is dissolved therein. The pH is adjusted to pH 5 and the solution made up to 10 l with water for injection. Following filtration, the solution is decanted into 100-ml brown-glass bottles and the vessels are sealed with suitable plastic caps.

The preparation and decanting are performed in pharmaceutical rooms under germ-free conditions.

### EXAMPLE 4

A high-dose sublingual spray for the treatment of antidiuretic disturbances and haemorrhagic diseases is prepared by dissolving 2 g desmopressin acetate, 0.1 g benzalkonium chloride, 9.115 g sodium chloride and 0.335 g malic acid in 950 g water for injection, adjusting the pH to 5.0 and making the solution up to 1 litre with water for injection. The solution is decanted into 50-ml brown-glass bottles with suitable plastic caps under germ-free conditions.

### EXAMPLE 5

A syrup for oral administration (with p-hydroxybenzoic acid methyl ester as the preservative) for the treatment of antidiuretic disturbances and haemorrhagic diseases is prepared by dissolving 100 g sorbitol, 1.5 g saccharin sodium and 1.675 g malic acid in 4.5 l purified water with stirring. 100 g desmopressin acetate and 10 g p-hydroxybenzoic acid methyl ester (previously dissolved in hot water) are then stirred into the solution and, after adjusting the pH to 5.0, made up to 5 l with water. The solution is decanted into 100-ml brown-glass bottles of hydrolytic class II.

The preparation is performed in germ-free pharmaceutical production rooms.

### EXAMPLE 6

A syrup for oral administration (with p-hydroxybenzoic acid methyl ester and p-hydroxy-benzoic acid propyl ester as the preservative) for the treatment of antidiuretic disturbances and haemorrhagic diseases is prepared by dissolving 60 g sorbitol, 0.9 g saccharin sodium, 60 mg desmopressin acetate and 1.005 g malic acid in 2.7 l purified water. 5.4 g p-Hydroxybenzoic acid methyl ester and 0.6 g p-hydroxybenzoic acid propyl ester (previously dissolved in hot water) are then added, the pH is adjusted to 5.0, and the solution made up to 3.0 l with purified water and decanted into suitable brown-glass bottles with plastic stoppers under germ-free conditions.

It has been shown that a change in the pH can occur if the glass of the glass vessels into which the solution is decanted is of inadequate quality. For this reason, within the framework of the invention it is advantageous if, when preparing a pharmaceutical substance containing a preparation according to the invention, the decanting is performed into glass vessels of hydrolytic class I or II, especially when the malic-acid concentration of the solution used is high.

The content of preservative mentioned in the preceding examples should be regarded purely as examples. Experiments have shown that the benzalkonium chloride content of the preparation can advantageously be between 0.05 and 0.20 mg/ml. Similarly within the framework of the invention it is perfectly possible for the content of preservative p-hydroxybenzoic acid methyl ester to be 1 to 2.5 mg/ml. Especially favourable values have been obtained in the region of 1 to 2 mg/ml. The content of the preservative p-hydroxybenzoic acid methyl ester may be combined with a content of p-hydroxybenzoic acid propyl ester, the latter content advantageously being between 0 and 0.2 mg/ml, preferably between 0.1 and 0.2 mg/ml.

The active substance (desmopressin) is advantageously used in a low concentration of around 0.005 to 2 mg/ml. For pharmaceutical substances intended for oral administration, a desmopressin content of 0.005 to 0.04 mg/ml has proved advantageous. For pharmaceutical substances for nasal administration the content is higher as a rule, about 0.02 to 2.0 mg/ml, preferably 0.08 to 1.0 mg/ml. In the case of pharmaceutical substances for sublingual administration, on the other hand, the desmopressin content is higher as a rule, around 0.4 to 2.0 mg/ml.

1. A stable pharmaceutical preparation for nasal, oral or sublingual administration to patients in the form of a liquid solution, in particular an aqueous solution of desmopressin as the active substance, this liquid solution containing an osmotic agent and a buffer which maintains the pH within the range 4 to 6, preferably at around 5, characterised in that the buffer used to stabilise the desmopressin is malic acid.

2. A preparation according to claim 1, characterised in that the malic-acid buffer is present in a low concentration, preferably in the region 1 to 5 mM, in particular around 2.5 mM.

3. A preparation according to claim 1 or 2, characterised in that the malic acid is present as the racemate.

4. A preparation according to one of claims 1 to 3, characterised in that the desmopressin is present in a low concentration, in particular within the concentration range 0.005 to 2 mg/ml.

5. A preparation according to claim 4, characterised in that, for a preparation intended for oral administration, the desmopressin is present in the concentration 0.005 to 0.04 mg/ml.

6. A preparation according to claim 4, characterised in that, for a preparation intended for nasal administration, the desmopressin is present in the concentration 0.02 to 2.0 mg/ml, preferably 0.08 to 1.0 mg/ml, in particular 0.1 mg/ml.

7. A preparation according to claim 4, characterised in that, for a preparation intended for sublingual administration, the desmopressin is present in the concentration 0.4 to 2.0 mg/ml.

8. A preparation according to one of claims 1 to 7, characterised in that NaCl is used to adjust the osmotic pressure.

9. A preparation according to one of claims 1 to 8, characterised in that another buffer is used in addition to malic acid, e.g. acetate/acetic acid.

10. A preparation according to one of claims 1 to 9, characterised in that it is free from preservatives.

11. A preparation according to one of claims 1 to 9, characterised in that the preparation contains 0.05 to 0.20 mg/ml benzalkonium chloride.

12. A preparation according to one of claims 1 to 9, characterised in that the preparation contains 1 to 2.5 mg/ml, preferably 1 to 2 mg/ml, in particular 2 mg/ml p-hydroxybenzoic acid methyl ester, if necessary in combination with up to 0.2 mg/ml, preferably with 0.1 to 0.2 mg/ml, in particular with 0.15 to 0.2 mg/ml p-hydroxybenzoic acid propyl ester.

13. A preparation according to one of claims 1 to 12, characterised in that it contains 0.1 mg/ml desmopressin acetate dissolved in water, DL-malic acid in a concentration of 2.5 mM, NaCl as the osmotic agent and, if necessary, 0.10 mg/ml benzalkonium chloride as the preservative, the pH of the preparation being kept at about 5.

14. A preparation according to one of claims 1 to 13, characterised in that the malic acid is present in the form of a salt, e.g. the sodium salt, in the dissolved state.

15. A method of preparing a pharmaceutical substance containing a preparation according to one of claims 1 to 14, in which the preparation is decanted into glass vessels of hydrolytic class I or II.

16. A pharmaceutical substance for nasal administration containing a preparation according to one of claims 1 to 14.

17. A pharmaceutical substance for oral administration containing a preparation according to one of claims 1 to 14.

18. A pharmaceutical substance for sublingual administration containing a preparation according to one of claims 1 to 14.

19. A method of treating a patient suffering from antidiuretic disturbances, in particular enuresis nocturna or diabetes insipidus, characterised in that a pharmaceutical substance according to claim 16, 17 or 18 is administered.

20. A method of treating a patient suffering from haemorrhagic diseases, such as haemophilia A, Willebrand-Jürgen's syndrome or postoperative bleeding, characterised in that a pharmaceutical substance according to claim 16, 17 or 18 is administered.
